(19) United States
(12) Patent Application Publication
(10) Pub. No.: US 2019/0029650 A1
(43) Pub. Date: Jan. 31, 2019

(54) ELASTOGRAPHY IMAGING WITH MAGNETIC RESONANCE IMAGING GUIDED FOCUSED ULTRASOUND

(71) Applicant: University of Utah Research Foundation, Salt Lake City, UT (US)

(72) Inventors: Dennis Parker, Salt Lake City, UT (US); Allison Payne, Salt Lake City, UT (US); Joshua De Bever, Salt Lake City, UT (US); Douglas Christensen, Salt Lake City, UT (US)

(21) Appl. No.: 16/070,237

(22) PCT Filed: Jan. 10, 2017
(86) PCT No.: PCT/US2017/012827

§ 371 Date: Jul. 13, 2018

Related U.S. Application Data
(60) Provisional application No. 62/278,879, filed on Jan. 14, 2016.

(51) Int. Cl. A61B 8/08; A61B 5/00; A61B 5/055; G01R 33/48; G01R 33/563

(52) U.S. Cl. CPC A61B 8/485; A61B 5/0035; A61B 5/055; A61B 2090/374; G01R 33/4804; G01R 33/4814; G01R 33/56358; A61B 8/5261

(57) ABSTRACT

A technology is described for multipoint tissue elastic property measurement. An example method (700) 700 includes generating a treatment map (710) of an anatomical region that shows focal points within the anatomical region to be exposed to Focused Ultrasound (FUS) pulses; acquiring a reference MR-ARFI image (720) of the anatomical region containing the focal points using the treatment map; acquiring an active MR-ARFI image (730) for each of the focal points in the anatomical region during exposure of the focal points to the FUS pulses using the treatment map; interleaving the reference MR-ARFI image and active MR-ARFI images (740) to create a combined image of the anatomical region and the focal points; and calculating a tissue displacement measurement (750) for the focal points exposed to the simultaneous and/or rapidly interleaved FUS pulses using the combined image of the anatomical region and the focal points exposed to the simultaneous FUS pulses.

### RELATED APPLICATIONS

This application claims priority to U.S. Provisional Application No. 62/278,879, filed Jan. 14, 2016 which is incorporated herein by reference.

### GOVERNMENT INTEREST

This invention was made with government support under Grant Nos. CA172787 and EB013433 awarded by the National Institutes of Health. The government has certain rights in the invention.

### BACKGROUND

Focused ultrasound (FUS) is a non-invasive mechanism of thermal therapy that can be guided by magnetic resonance imaging (MM) or ultrasound (US) imaging. Both MRI guided FUS (MRgFUS) and US guided FUS (USgFUS) can accurately measure displacement caused by acoustic radiation force of an ultrasound beam. USgFUS can monitor tissue elastic properties (elastic modulus and shear wave velocity) before, after, and dynamically during treatment. MRgFUS can monitor temperature rise and thermal dose in relation to the clinical standard of tissue death in thermal ablation therapies. However, limitations in these technologies remain: For example, USgFUS cannot measure temperature while MRgFUS cannot efficiently make images of tissue elastic properties.

### SUMMARY

A technology is described for obtaining elastic tissue displacement measurements at a plurality of focal points in an image volume. The elastic tissue displacement measurements may provide the ability to remotely palpate an ablated tissue volume and monitor the formation of lesions created using FUS pulses. Monitoring lesion formation may increase the efficiency of MRgFUS ablation procedures by improving the accuracy in estimating the tissue volume that has been effectively treated.

In one example, a reference MR-ARFI image of an anatomical region that is to be treated using FUS pulses can be captured. The reference MR-ARFI image can provide a phase reference for focal points that are to be exposed to the FUS pulses. Thereafter, active MR-ARFI images for focal points located in the anatomical region can be captured during exposure of the focal points to the FUS pulses. The active MR-ARFI images can be interleaved with the reference MR-ARFI image to create a combined image of the anatomical region containing the focal points. A tissue displacement measurement can then be calculated for the focal points exposed to the FUS pulses using the combined image of the anatomical region, and the tissue displacement measurement can be used to monitor treatment of the tissue included in the anatomical region.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a block diagram that illustrates an example system for measuring multipoint displacement of tissue exposed to FUS pulses.

FIG. 2 is a graphic illustrating an example of multipoint Magnetic Resonance Acoustic Radiation Force Impulse (MR-ARFI) with multipoint spacing.

FIGS. 3a-3b shows pulse sequences used for (a) gradient echo MR-ARFI and (b) spin echo MR-ARFI. MR-ARFI works by applying motion-encoding gradients (MEG) during a short FUS pulse such that the image phase becomes a function of the tissue displacement. A 3D displacement pattern measured using a hydrogel phantom FIG. 3c is shown in FIG. 3d and FIG. 3e for axial and coronal cross-sections, respectively. Images are interpolated to 0.5-mm voxel spacing (distance scale on axes is in mm).

FIG. 4 is a graphic that illustrates an example 3D simulation of multipoint MR-ARFI acquisition.

FIGS. 5a-5c include graphics illustrating example multipoint MR-ARFI measurements.

FIG. 6 is a block diagram that illustrates an example of k-space data sharing in multipoint MR-ARFI.

FIG. 7 is a flow diagram that illustrates an example method for multipoint tissue displacement measurement.

FIG. 8 is block diagram illustrating an example of a computing device that may be used to execute a method for multipoint tissue displacement measurement.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

Definitions

In describing and claiming the present invention, the following terminology will be used.

The singular forms “a,” “an,” and “the” include plural referents unless the context clearly dictates otherwise. Thus, for example, reference to “a particle” includes reference to one or more of such materials and reference to “subjecting” refers to one or more such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of” For example, “at least one of A,B and C” explicitly includes only A, only B, only C, and combinations of each (e.g. A+B, B+C, A+C, and A+B+C).

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Elastographic Imaging

In the case of thermal ablation, treatment efficacy is defined in terms of the desired cell death. The relative effects of thermal therapies are usually estimated in terms of thermal dose, which is a non-linear function of temperature. However, several different acute tissue states can result in eventual cell death and it is difficult to consistently determine when the endpoint is reached. The uncertainty in treatment outcome leads to the possibility of an ineffective under-treatment or an over-treatment that is effective but may damage surrounding normal tissues. More accurate treatment outcome assessment would make it possible to successfully execute the desired treatment while minimizing the damage to normal tissues.

Thermal dose: The thermal dose metric has been developed in an attempt to standardize the effects of thermal treatments with different temperature distributions and time histories. Although, there have been several methods studied to measure temperature with ultrasound, challenges remain that keep ultrasound from being used to monitor temperature in FUS treatments. Mill can measure temperature change in aqueous tissues but not in lipids and requires knowledge of the starting baseline temperature.

Tissue elasticity: Tissue mechanical properties, such as elasticity or stiffness, change with thermal ablation. At high thermal dose, cells can become completely coagulated and water can be removed resulting in greatly increased stiffness. Both ultrasound and MRI elastography methods have shown that tissue elasticity changes with temperature and changes irreversibly at even low levels of thermal dose. Advanced diagnostic ultrasound systems can rapidly image tissue displacement and shear waves produced by the radiation force of short, intense, focused ultrasound pulses and can detect changes in shear wave velocity due to thermal ablation procedures. MR-elastography, using an external source to induce shear waves in the tissue and phase contrast displacement measurement methods, has been used to assess changes in elasticity in conjunction with thermal ablation. In a study using FUS in ex vivo bovine tissue, the elastic shear modulus was found to increase with ablation and the increase was nearly linear in ultrasound energy deposited. Implementing diagnostic ultrasound or MR-elastography tissue vibrators in conjunction with MRgFUS introduces logistical challenges.

Focused ultrasound (FUS) is a non-invasive interventional technique that focuses ultrasound energy deep within the body to quickly heat and cause necrosis in tumors or other diseased tissues while sparing the surrounding normal tissues. FUS has been used to treat benign and malignant solid tumors, as well as a number of neurological pathologies, including movement disorders, epilepsy, chronic pain, and psychiatric disorders. As a new treatment option for breast cancer, FUS yields improved cosmetic outcomes by maintaining breast architecture and avoiding scarring. If treatment outcomes can be more accurately assessed, FUS can offer a non-invasive option for treating localized primary tumors, avoid the discomfort and potential complications associated with general anesthesia and surgery, and be a viable treatment alternative when radiation and chemotherapy limits have been reached.

MRI has the unique capability to obtain high-resolution 3D images of healthy and diseased tissues for diagnosis, treatment planning, guidance and assessment of thermal therapy procedures. MRI can also image temperature change over the treatment volume allowing determination of thermal dose and facilitating real-time control, safety monitoring, and online treatment optimization. Several other MRI parameters change with the state of the tissue. The MRI water diffusion coefficient, MRI relaxation times T1 (longitudinal) and T2 (transverse), and the exchange rate between free and protein bound water depend on the temperature as well as the state of water in the tissue. Thermal therapy can change the amount of water in the tissue, resulting in irreversible changes in these MRI parameters. Finally, exogenous contrast can be used in MRI to interrogate the state of the vascular supply to a tumor, which can be affected by thermal therapy.

To assist FUS application as described herein, MRI can obtain high-resolution 3D images of healthy and diseased tissues for diagnosis, treatment planning and assessment. MRI can also image temperature change over the treatment volume allowing determination of thermal dose (which predicts tissue necrosis) and facilitating real-time control, safety monitoring, and online treatment optimization.

Some FUS systems use diagnostic ultrasound for guidance, combining the therapeutic and diagnostic transducers into one unit. Although ultrasound cannot monitor temperature accurately or generate 3D images comparable to MM, it can provide a real-time view of the FUS focus. Advanced diagnostic ultrasound systems can rapidly image the tissue displacement and shear waves produced by the radiation force of short, intense, focused ultrasound pulses. The acoustic radiation force density, Γₐ, is given by

$${Γ}_{a} = \frac{2 α I}{c}$$

where I is the acoustic intensity, a is the pressure absorption coefficient, and c is the speed of sound. Assuming linear tissue response, the resulting displacement (w) is expected to be linear with the applied force: w∝Γₐ/μ where μ, the local Lamé shear constant, is a measure of tissue stiffness. MRI methods using motion encoding gradients synchronized with a short FUS pulse can make 2D or 3D images of the tissue displacement caused by the radiation force.

Although Magnetic Resonance Acoustic Radiation Force Impulse (MR-ARFI) may be unable to measure spatial distribution of tissue elastic properties, MR-ARFI imaging can produce 2D and 3D images of acoustic radiation force applied at a single point. This MR-ARFI map showing the position and quality of the focal spot can be complementary to MR thermometry and can be useful during all aspects of MRgFUS therapy. MR-ARFI sequences have been used for both beam localization and adaptive focusing. The technology includes 3D MR-ARFI methods that image the 3D single-point tissue displacement fields and includes analytic models of this field (see de Bever J, Todd N, Odeen H, Parker D L. Evaluation of a 3D MR Acoustic Radiation Force Imaging Pulse Sequence Using a Novel Unbalanced Bipolar Motion Encoding Gradient. Magn Reson Med 2015, which is incorporated herein by reference). 3D acquisition allows band-limited interpolation to accurately locate the ultrasound focus and the resulting displacement distribution in 3D. Despite the ability of MRI to make 2D and 3D images of single point displacement fields, efficient methods to image tissue elastic properties at more than a single point are lacking.

Despite the great potential of focused ultrasound in non-invasive therapy, treatment endpoint assessment has been a challenge. Subject motion causes temperature measurement artifacts that add inaccuracy to thermal dose as an endpoint measure. MRI images such as T2w (weighted images) or late Gadolinium enhanced (LGE) images made acutely after treatment may not be specific to tissue that will become necrotic. Diagnostic ultrasound, which can measure tissue elastic properties, can require removing the patient from an MRI scanner and using a hand-held probe, both leading to registration problems. The ability to make efficient, multipoint measurements of tissue elastic properties with MRI may be a highly important adjuvant assessment of the state of tissue before and after treatment.

The methods described herein can expand the current capabilities of MR-ARFI by enabling MRI to evaluate changes in tissue elastic properties over a large area instead of at a single point. MRgFUS is a single modality that can both generate a therapeutic effect and assess tissue damage with both thermal and mechanical mechanisms. Simultaneous displacement and temperature change measured by the developed mpMR-ARFI protocol can be used to estimate an effective tissue elasticity and to track the changing tissue properties with temperature and thermal dose over time demonstrating the potential clinical utility of the technique. The effective elasticity parameter can also be correlated to the independently measured Lame shear constant in ex vivo tissues. The effective elasticity can be proportional to the ratio of displacement to temperature increase.

Furthermore, this approach allows elastic displacement measurements at a plurality of points in the image volume to be obtained, thereby effectively acquiring a low resolution image of tissue elasticity in the same time that conventional MR-ARFI measures displacement at a single point. Volumetric tissue elasticity measurements made before and between sonications can provide the ability to perform what may be called remote palpation of the evolving thermal lesion formed by the MRgFUS treatment at multiple positions and times during the procedure. This can increase the amount of information available to determine a successful outcome.

The FUS pulses can be either or both simultaneous and temporally interleaved with each TR and between TR's. Each TR is a repetition time (usually about 30 to 50 msec), and a 3D image generally uses many of these TR's (maybe 128×32). Each 3D volume is acquired with multiple sets of measurements (multiple TR's) and for each instance of the 3D volume, one or more FUS pulses are interleaved by:

Pushing multiple points in the same TR (and therefore in the same instance of the 3D volume) by:

1) applying a single pulse with 2 or more focal points (this may be simultaneous, but 2 to 4 points in the same pulse can be used);

2) by switching rapidly (about 5 msec) between 2 or more focal positions; or

3) both 1) and 2);

Pushing different sets of points in the subsequent interleaved instances of the 3D volume.

Multiple focused ultrasound (FUS) pulses can also be temporally interleaved during a single MM acquisition, decreasing total acquisition time and reducing tissue heating compared to acquiring the same points sequentially with single-point MR-ARFI (spMR-ARFI). Acquisition time can be further reduced by focusing at two or more points in each motion encoding interval.

The multi-point MR-ARFI can provide a method to assess MRgFUS treatment endpoint that is complementary to thermal dose and LGE images. By measuring tissue displacement as a function of ultrasound intensity and changes in displacement during the procedure, it can provide the ability to remotely palpate the ablated volume to monitor the formation of lesions created with MRgFUS. This independent monitoring of lesion formation can increase the efficiency of MRgFUS ablation procedures by improving the accuracy in estimating the tissue volume that has been effectively treated.

The technology can involve modifications to a focused ultrasound controller, a MR-ARFI pulse sequence, and new reconstruction methods. MR-ARFI works by applying gradients during a short FUS pulse such that the image phase becomes a function of the tissue displacement. In spin echo (SE) MR-ARFI phase does not depend on temperature, but the refocusing RF pulse used may include long delays between RF pulses (long TR) to avoid reduced image signal and compromised ARFI measurement accuracy. Gradient echo (GRE) MR-ARFI allows short TR (˜50ms), but the image phase may be a function of both temperature and displacement. By acquiring measurements at multiple echo times (TE), phase contributions due to temperature, which may be a function of TE, can be separated from measurements resulting from tissue displacement. Because overly rapid FUS pulsing at a single point may cause unwanted tissue heating, interleaving of the acquisition of points (e.g., 12 to 24) spaced several mm apart can be performed. Interleaving and data sharing may decrease acquisition time and reduce tissue heating compared to single point MR-ARFI.

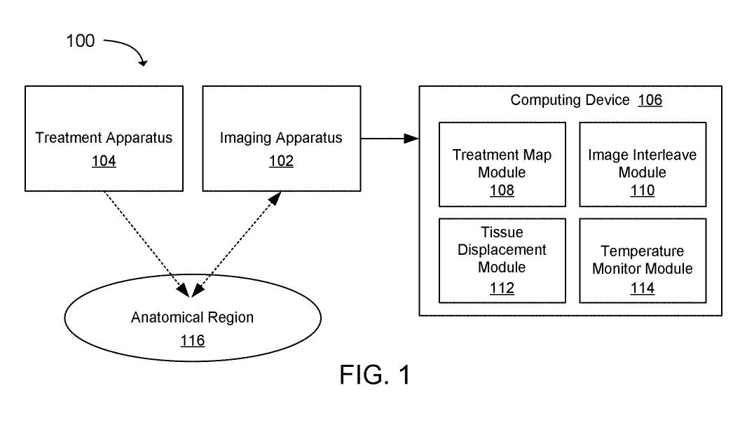

FIG. 1 is a block diagram illustrating an example system 100 for measuring multipoint tissue displacement of tissue exposed to FUS pulses. As illustrated the system can include a computing device 106 that is in communication with an imaging apparatus 102. The imaging apparatus 102 can include an MRI apparatus. In one example, the MRI apparatus may be configured to use a 3D MR-ARFI spin echo technique to capture images. In another example, the MRI apparatus may be configured to use a 3D MR-ARFI gradient echo technique to capture images. The imaging apparatus 102 can be operable in conjunction with a treatment apparatus 104 to acquire a reference image of an anatomical region 116 containing focal points prior to exposing the focal points to FUS pulses. The imaging apparatus 102 in conjunction with the treatment apparatus 104 can also be used to acquire an active image for each of the focal points in the anatomical region 116 during exposure of the focal points to the simultaneous and/or rapidly interleaved FUS pulses.

In one example, the treatment apparatus 104 can include an ultrasound transducer configured to emit FUS pulses at tissue represented by focal points within the anatomical region 116. In another example, the ultrasound transducer can be configured to emit a pressure pattern that simultaneously focuses on two or more focal points within the anatomical region 116. A focal point can be a predetermined volume within the anatomical region 116 that has been selected to receive treatment via one or more FUS pulses.

The computing device 106 can be configured to create a combined image of the anatomical region 116 that includes the focal points. The combined image can be created by interleaving a reference image of the anatomical region 116 and active images that include the focal points in the anatomical region 116. Displacement measurements for the focal points exposed to the FUS pulses can be calculated using the combined image. The computing device 106 can include modules used to create the combined image and calculate displacement measurements. The modules may include a treatment map module 108, an image interleave module 110, a tissue displacement module 112, a temperature monitor module 114, and other modules and/or services.

The treatment map module 108 can be configured to generate a map of an anatomical region 116 (i.e., a treatment map) that includes focal points within the anatomical region 116 to be exposed to simultaneous and/or rapidly interleaved FUS pulses. In one example, an anatomical region MR-ARFI image of an anatomical region 116 can be acquired using the MRI apparatus 102. Focal points that are to be exposed to FUS pulses can be overlaid on the anatomical region MR-ARFI image. As an illustration, a user can add focal points to the anatomical region MR-ARFI image displayed in a graphical user interface (not shown) by selecting a region that is to be treated and adding a graphical representation to the image that represents the focal point. The focal points mark tissue that is to be ablated using FUS pulses. In one example, the treatment map module 108 may be configured to generate a three-dimensional (3D) map of an anatomical region 116 that includes focal points to be exposed to FUS pulses.

The image interleave module 110 can be configured to interleave a reference MR-ARFI image (e.g., a treatment map generated using the treatment map module 108) and active MR-ARFI images to create a combined image of an anatomical region 116 that includes focal points. The reference MR-ARFI image can provide a phase reference for the focal points to be exposed to FUS pulses. An active MR-ARFI image for each of the focal points in the anatomical region 116 can then be acquired during exposure of the focal points to the FUS pulses. That is, the active MR-ARFI image can be acquired using the imaging apparatus 102 during treatment of the anatomical region 116. The reference MR-ARFI image and active MR-ARFI images can then be interleaved to create a combined image of the anatomical region 116 that includes imaging data for the focal points being treated using FUS pulses.

The tissue displacement module 112 can be configured to calculate a tissue displacement measurement for focal points exposed to FUS pulses using a combined image of an anatomical region 116 created using the image interleave module 110 as described above. The tissue displacement measurement may be a function of FUS pulse intensity and changes in tissue displacement during exposure of tissue to a FUS pulse. For example, a thermal dose delivered by FUS pulses to tissue located at the focal points can be measured in tandem with measuring tissue displacement of tissue being exposed to the FUS pulses, and the measurements can be used to determine the tissue displacement measurement.

In one example, the temperature monitor module 114 can be configured to monitor the temperature in the anatomical region 116 being exposed to the FUS pulses and provide the temperature to the tissue displacement module 112 which uses the temperature to calculate a tissue displacement measurement. In one example, the temperature may be calculated by performing baseline subtraction between a sequence of MR-ARFI reference images.

The tissue displacement module 112 can be configured to assess tissue damage occurring at focal points according to a tissue displacement measurement and determine whether a treatment endpoint has been realized based in part on the tissue displacement measurement. For example, the tissue displacement module 112 may monitor tissue damage by detecting and monitoring the formation of lesions during treatment as explained in greater detail later.

The computing device 106 can comprise a processor-based system. The various processes and/or other functionality may be executed on one or more processors that are in communication with one or more memory modules. In one example, a number of computing devices 106 may be arranged in one or more server banks or computer banks or other arrangements. The computing device 106 may be connected to a network. The network may include any useful computing network, including an intranet, the Internet, a local area network, a wide area network, a wireless data network, or any other such network or combination thereof. Components utilized for such a system may depend at least in part upon the type of network and/or environment selected. Communication over the network may be enabled by wired or wireless connections and combinations thereof.

FIG. 1 illustrates that certain processing modules may be discussed in connection with this technology and these processing modules may be implemented as computing services. In one example configuration, a module may be considered a service with one or more processes executing on a server or other computer hardware. Such services may be centrally hosted functionality or a service application that can receive requests and provide output to other services or consumer devices. While FIG. 1 illustrates an example of a system 100 that can implement the techniques above, many other similar or different environments are possible. The example environments discussed and illustrated herein are merely representative and not limiting.

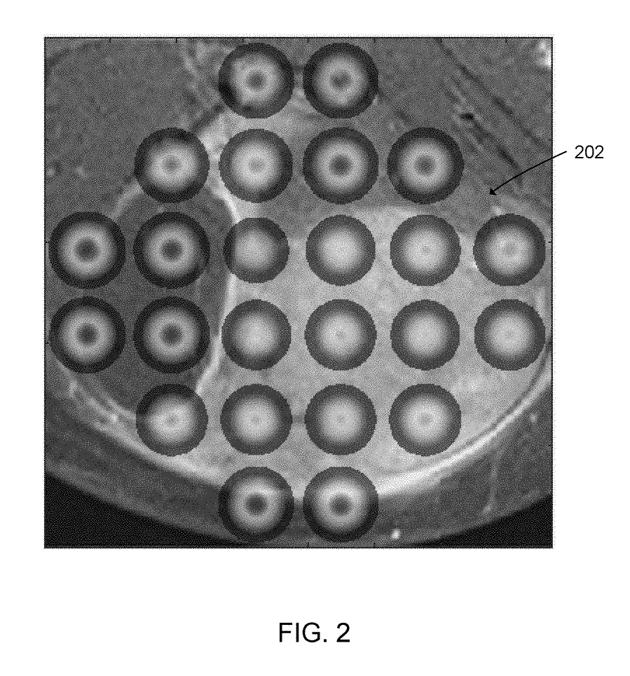

FIG. 2 illustrates a graphic of an example multipoint MR-ARFI with 5 mm spacing and analysis of the displacement pattern between points. MRgFUS images of elastic displacement can be generated, providing preliminary demonstration of MRgFUS elastography. The 24 points (spaced by 5 mm) illustrated in FIG. 2 cover a 3 cm diameter steering range of a MRgFUS system.

FUS control software can be modified to allow a trajectory of short FUS pulses. To increase the number of points excited, the phases of the multiple-element phased array FUS transducer can be adjusted to focus (and perform ARFI displacement) simultaneously at two or more well separated points. Because phased-array ultrasound transducers can rapidly steer between different focal points, multiple points can be interrogated during a single acquisition interval. The power of phased-array transducers can be used to create volumetric ablation points through creating simultaneous focal spots or through rapid electronic scanning of a single focal point. A 3D Gradient echo MR-ARFI/temperature sequence can be used that interleaves multipoint acquisition with data sharing. As an illustration, if N_{fz} focal positions can be acquired in a multipoint ARFI map (e.g. N_{fz}=24), then the same 3D volume can be acquired N_{fz} times with FUS pulses (ON images) and once without FUS pulses (OFF images) (e.g. N_{fz}+1=25). The OFF image serves as the phase reference image for all of the ON images. Because the displacement pattern may be broad in the ultrasound propagation (slice encoding) direction, acquisition time can be reduced or N_{fz} increased by only acquiring a few central slice encoding measurements (|k_{z}<k_{cent}) for the N_{fz} ON images. The outer slice encodings (k_{cent}<|k_{z}|<N_{z}/2) for all ON images can be obtained from the OFF image. OFF image acquisition can be interleaved with the ON image acquisitions to further reduce tissue heating.

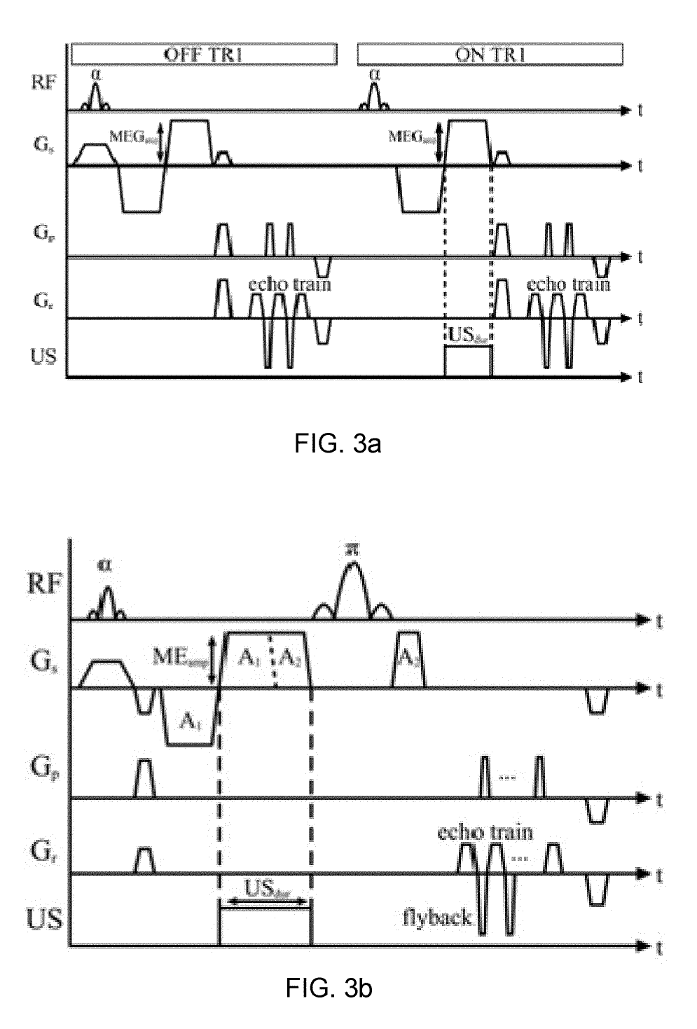

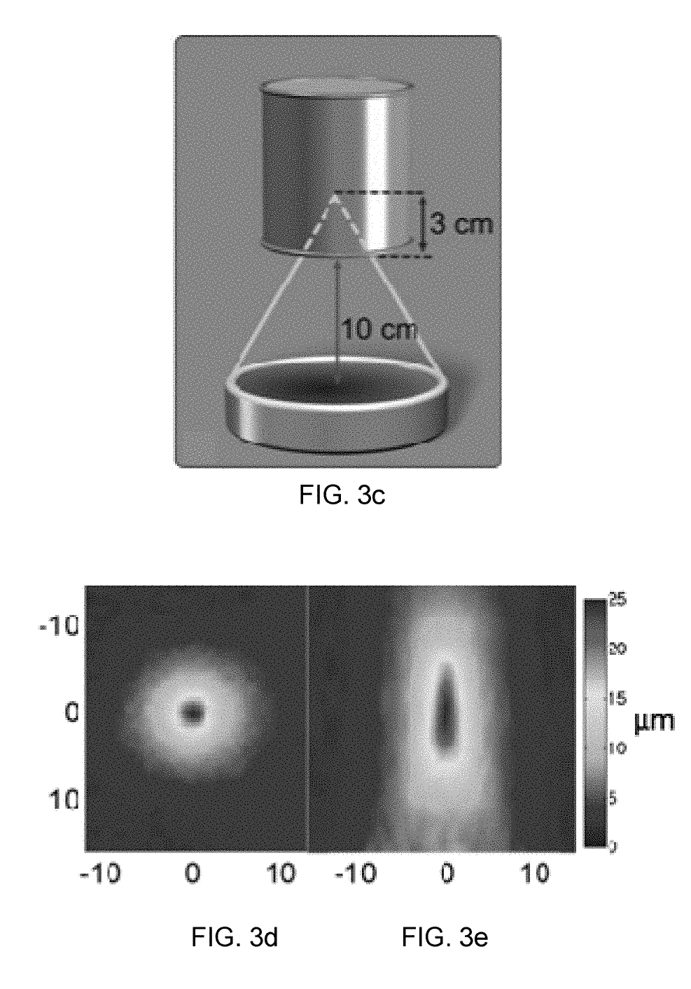

In one specific example, FIG. 3a shows pulse sequences used for gradient echo MR-ARFI and FIG. 3b shows pulse sequences used for spin echo MR-ARFI. MR-ARFI works by applying motion-encoding gradients (MEG) during a short FUS pulse such that the image phase becomes a function of the tissue displacement. A 3D displacement pattern measured using a hydrogel phantom (shown in FIG. 3c) is shown in FIG. 3d and FIG. 3e for axial and coronal cross-sections, respectively. Images are interpolated to 0.5-mm voxel spacing (distance scale on axes is in mm).

The technology includes 3D MR-ARFI in both spin echo and gradient echo implementations (FIGS. 2A-2B). When the ultrasound pulses are repeated, as they are in both 2D and 3D MR-ARFI methods, a small, but measurable increase in temperature occurs during the sequence. In spin echo (SE) MR-ARFI (FIG. 2B), phase does not depend on temperature, but because the refocusing RF pulse inverts the residual longitudinal magnetization, long delays are required between excitation RF pulses (long TR) to avoid reduced image signal and compromised ARFI measurement accuracy. Gradient echo (GRE) MR-ARFI allows short TR (˜50 ms), but the image phase is a function of both temperature and displacement. By interleaving identical measurements with ultrasound off and on (FIG. 3a) or by acquiring measurements at multiple echo times (TE), phase contributions due to temperature, which may be a function of TE, can be separated from those resulting from tissue displacement. Because temperature increase is slow compared to the interleaved measurement length, the interleaved OFF image has nearly identical phase to the ON image and the temperature-dependent phase can be removed by complex subtraction (which also removes coil phase and any other constant phase sources). In addition, interleaving an OFF acquisition increases the time between FUS pulses and thereby reduces heating.

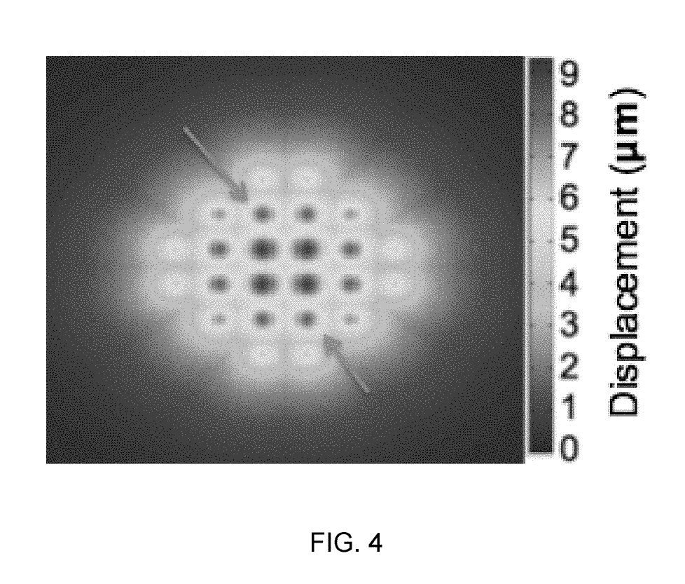

FIG. 4 provides a specific example of a 3D Simulation of a combined 24-point mpMR-ARFI acquisition. The 24 points are spaced 5 mm apart, utilizing a phased-array FUS transducer's full 3 cm diameter steering volume. In this example, a complete image volume can be obtained for each point in the multipoint MR-ARFI acquisition. Merging of points into a single image display may be done algorithmically as desired. In this case each point in the composite displacement image was formed by taking the maximum displacement at the corresponding point over all 24 individual displacement images. Arrows indicate example points that could be excited simultaneously.

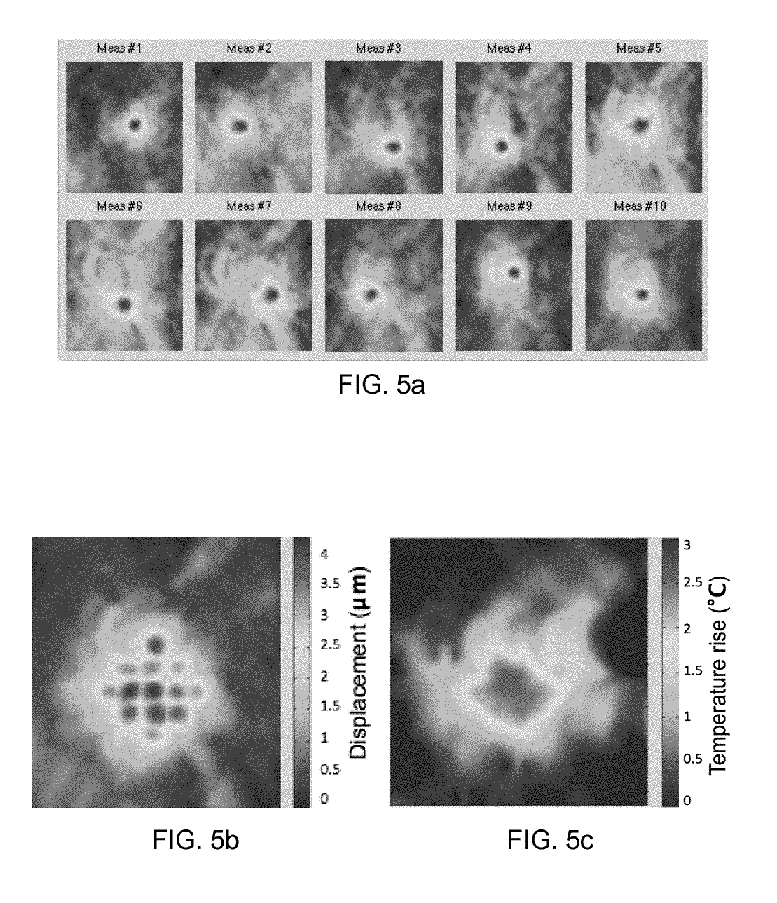

The FUS controller and the 3D MR-ARFI sequence were modified to perform interleaved acquisition of multiple points. FIG. 5a-FIG. 5c provide images of 3D MR-ARFI displacement maps from a multipoint MR-ARFI sequence obtained by pushing on one point in each instance of the 3D volume. A FUS controller and a 3D MR-ARFI sequence are used to perform interleaved acquisition of multiple points. The results acquired at 3 Tesla with segmented echo planar (EPI), TR/TE=100/36 ms, EPI=7, matrix=128×104×20, FOV=160×130×40 mm, interpolated to 0.5 mm isotropic voxel spacing, total acquisition time=546 seconds or 39 seconds per measurement point.

Image reconstruction can be performed by combining the outer slice encoding OFF image measurements with the data of each of the N_{f}, ON images to form high-resolution images for each FUS point. Because the same volume may be imaged many times (e.g., as many as 25 times), and because only the image phase may be expected to change between acquisitions, magnitude averaging can be incorporated for increased image SNR (Signal-to-Noise Ratio). The phase for temperature and displacement for each FUS point may be determined from each ON image separately. Also, displacement modeling can be used to obtain elasticity distributions around each point.

In one example, to shorten acquisition time: 1) TR can be reduced by at least a factor of two because tissue heating is spread by interleaving; 2) Because the displacement pattern is broad in the propagation direction, only central lines in k_{z} can be acquired and the outer lines can be obtained from the single OFF image; 3) Fractional phase and slice encoding can be applied; and 4) Because the same volume is being acquired multiple times with only differences in the phase distribution, undersampling with constrained reconstruction can be used to improve efficiency. Taken together the efficiencies listed can achieve an acceleration factor of 24 (2×2×3/2×2). The same efficiencies applied to spMR-ARFI can achieve an approximate acceleration factor of 4 (2×4/3×3/2×1), giving mpMR-ARFI a 6-fold gain in efficiency over spMR-ARFI.

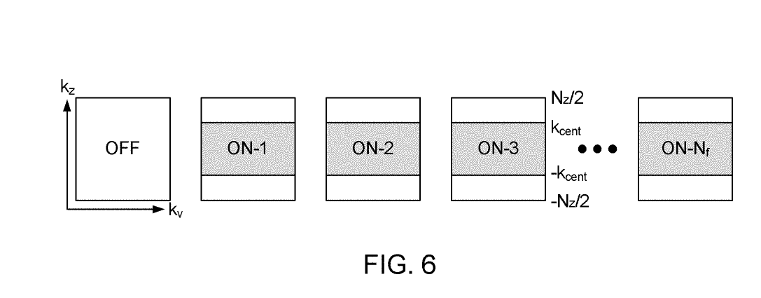

FIG. 6 illustrates k-space data sharing in multipoint MR-ARFI. Shading is used in FIG. 6 to represent data sharing. Central k_{z} kspace lines can be acquired for ON images, interleaved with the central k_{z} kspace lines for an OFF image. The outer OFF k-space can be used for all ON images. As shown in FIG. 6, 3D GRE MR-ARFI/temperature sequence and image reconstruction can be performed to interleave multipoint acquisition with data sharing. If N_{f} focal positions are acquired in the multipoint ARFI map, then the same 3D volume is acquired N_{f} times with FUS pulses ON and once with FUS pulses OFF (i.e. total acquisitions =N_{f}+1). The OFF image serves as the phase reference image for all of the ON images. Because the displacement pattern may be broad in the ultrasound propagation (slice encoding) direction, acquisition time may be reduced (or N_{f} increased) by only acquiring a few central slice-encoding measurements (|k_{z}|<k_{cent}) for the N_{f} ON images. The outer slice encodings (k_{cent}<|k_{z}|<N_{z}/2) for all ON images can be shared from the OFF image. Central k-space for the OFF image acquisition can be interleaved with the same lines of the ON acquisitions to determine that the temperature phase in the OFF image matches that of the ON images.

Because the same volume is imaged many times (e.g., 25 times for FIG. 4), and because the image phase is expected to change between acquisitions, signal magnitude constraints can be applied across all images for increased image SNR. The phase for temperature and displacement for each FUS point can be determined from each ON image separately.

In some aspects, small phase variation may occur between the multiple interleaved ON images. One option is to interleave one or more additional OFF images to increase the background phase similarity between ON and OFF images. If a single interleaved OFF image does not adequately match the phase of all ON images, then one can either interleave more OFF images, or eliminate the cause of the unwanted phase difference. Stimulated echoes may also result in spurious phase contributions from sequential FUS pulses and can be determined by testing for eddy currents, rewinder gradients errors, and incomplete spoiling. Such echoes can be reduced or eliminated by reducing MRI flip angle or increasing TR. If TR is increased, at least 2 points per MEG can be maintained.

Simultaneous volumetric temperature and displacement measurements can depend differently on the tissue elasticity and thermal properties: w∝2αI/μc and ΔT∝αI. Because there may be issues of relative timing of measurement, as well as blurring by the ARFI Green's function and by thermal diffusion, one cannot solve for μ directly. However, the independence does allow qualitative discrimination between changes in absorption and elasticity. Accordingly, an effective elasticity parameter μ_{eff}∝2ΔT/cw can be used as a metric to correlate with clinical ablation.

The described volumetric mpMR-ARFI can confirm thermal dose endpoints to remotely palpate a volumetric ablation procedure at multiple time points during the ablation procedure. From these mpMR-ARFI displacement and temperature measurements before and after application of MRgFUS ablation, with 3D dynamic temperature distribution monitored by 3D MRTI, the distribution of changes in tissue displacement, temperature, and μ_{eff} can be correlated with the corresponding distribution of thermal dose. The manual palpability of the lesions in the ex vivo tissues can be correlated to changes in tissue stiffness as assessed with mpMR-ARFI and bench measurements. MRgFUS ablation-induced elasticity changes can also be correlated to the accumulated thermal dose as obtained by MRTI.

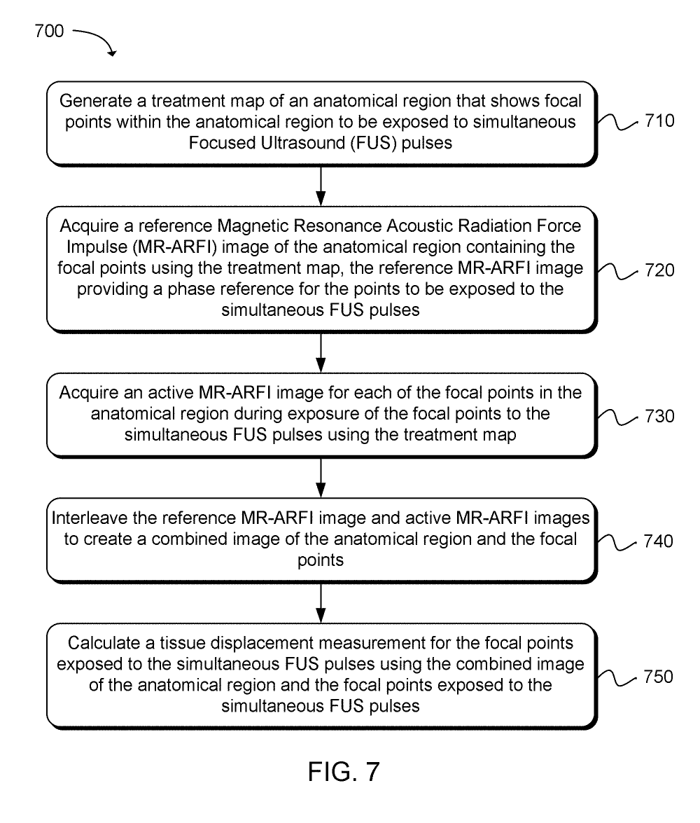

FIG. 7 is a flow diagram illustrating an example method 700 for multipoint tissue displacement measurement. The method can be executed as instructions on a machine, where the instructions are included on at least one computer readable medium or one non-transitory machine readable storage medium. The method can include the operation of generating a treatment map of an anatomical region that shows focal points within the anatomical region to be exposed to simultaneous Focused Ultrasound (FUS) pulses, as in block 710. The method can include the operation of acquiring a reference Magnetic Resonance Acoustic Radiation Force Impulse (MR-ARFI) image of the anatomical region containing the focal points using the treatment map, the reference MR-ARFI image providing a phase reference for the points to be exposed to the simultaneous FUS pulses, as in block 720. The method can include the operation of acquiring an active MR-ARFI image for each of the focal points in the anatomical region during exposure of the focal points to the simultaneous FUS pulses using the treatment map, as in block 730. The method can include the operation of interleaving the reference MR-ARFI image and active MR-ARFI images to create a combined image of the anatomical region and the focal points, as in block 740. The method can include the operation of calculating a tissue displacement measurement for the focal points exposed to the simultaneous FUS pulses using the combined image of the anatomical region and the focal points exposed to the simultaneous FUS pulses, as in block 750.

In one example, a change in elasticity may be obtained by combining a change in temperature (i.e., an increase in temperature) and a change in tissue displacement prior to exposing focal points to FUS pulses and after exposing the focal points to the FUS pulses. If tissue properties remain substantially static, and if the FUS pulse has the same intensity, then the temperature change with the FUS pulse (temperature rise) and the tissue displacement due to the FUS pulse may be the same before the treatment and after the treatment. However, the treatment likely changes the elasticity of the tissue and may also change an absorption coefficient (e.g., absorb more strongly after the treatment). If the tissue absorbs more strongly and is stiffer, the effects may compensate for the displacement and the effects may show the same displacement, even though the tissue has changed. However, any difference in the temperature increase can be used to tell whether more or less energy is being absorbed before or after the treatment. If the temperature increase is greater after the treatment, then the absorption coefficient increased. Thus, the ratio of displacement to temperature increase gives an estimate of effective elasticity. If the ratio changes, this indicates that the elasticity has changed. Namely, the temperature increase may be primarily related to the FUS energy absorbed by the tissue and the measured tissue displacement may be due to both the FUS energy absorbed and the tissue elastic property. By taking the ratio of the temperature increase and the displacement, the energy absorption can be eliminated as a factor in the elasticity change estimate.

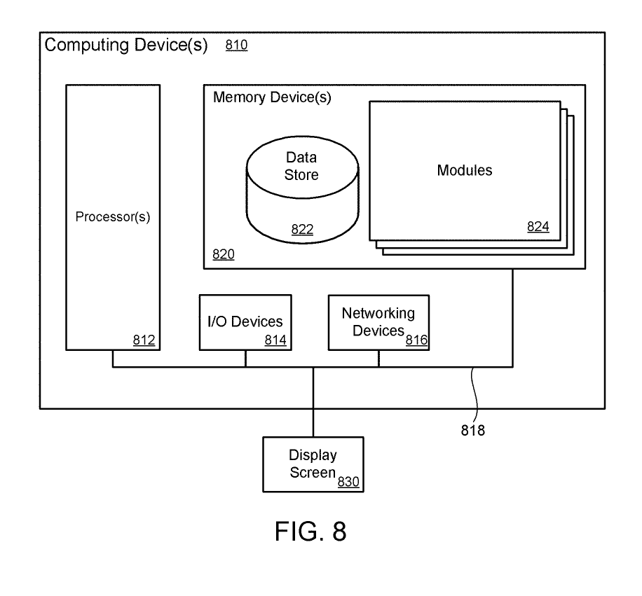

FIG. 8 illustrates a computing device 810 on which modules of this technology may execute. A computing device 810 is illustrated on which a high level example of the technology may be executed. The computing device 810 may include one or more processors 812 that are in communication with memory devices 820. The computing device 810 may include a local communication interface 818 for the components in the computing device. For example, the local communication interface 818 may be a local data bus and/or any related address or control busses as may be desired.

The memory device 820 may contain modules 824 that are executable by the processor(s) 812 and data for the modules 824. For example, the memory device 820 may contain a treatment map module, an image interleave module, a tissue displacement module, a temperature monitor module, and other modules. The modules 824 may execute functions that perform the methods described earlier. A data store 822 may also be located in the memory device 820 for storing data related to the modules 824 and other applications along with an operating system that is executable by the processor(s) 812.

Other applications may also be stored in the memory device 820 and may be executable by the processor(s) 812. Components or modules discussed in this description that may be implemented in the form of software using high programming level languages that are compiled, interpreted or executed using a hybrid of the methods.

The computing device may also have access to I/O (input/output) devices 814 that are usable by the computing devices. An example of an I/O device is a display screen 830 that is available to display output from the computing devices. Other known I/O devices may be used with the computing device as desired. Networking devices 816 and similar communication devices may be included in the computing device. The networking devices 816 may be wired or wireless networking devices that connect to the internet, a LAN, WAN, or other computing network.

The components or modules that are shown as being stored in the memory device 820 may be executed by the processor(s) 812. The term “executable” may mean a program file that is in a form that may be executed by a processor 812. For example, a program in a higher level language may be compiled into machine code in a format that may be loaded into a random access portion of the memory device 820 and executed by the processor 812, or source code may be loaded by another executable program and interpreted to generate instructions in a random access portion of the memory to be executed by a processor. The executable program may be stored in any portion or component of the memory device 820. For example, the memory device 820 may be random access memory (RAM), read only memory (ROM), flash memory, a solid state drive, memory card, a hard drive, optical disk, floppy disk, magnetic tape, or any other memory components.

The processor 812 may represent multiple processors and the memory device 820 may represent multiple memory units that operate in parallel to the processing circuits. This may provide parallel processing channels for the processes and data in the system. The local interface 818 may be used as a network to facilitate communication between any of the multiple processors and multiple memories. The local interface 818 may use additional systems designed for coordinating communication such as load balancing, bulk data transfer and similar systems.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions and may even be distributed over several different code segments, among different programs and across several memory devices. Similarly, operational data may be identified and illustrated herein within modules and may be embodied in any suitable form and organized within any suitable type of data structure. The operational data may be collected as a single data set, or may be distributed over different locations including over different storage devices. The modules may be passive or active, including agents operable to perform desired functions.

The technology described here may also be stored on a computer readable storage medium that includes volatile and non-volatile, removable and non-removable media implemented with any technology for the storage of information such as computer readable instructions, data structures, program modules, or other data. Computer readable storage media include, but is not limited to, non-transitory media such as RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which may be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology. The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A system for multipoint tissue elastic property measurement comprising:
an imaging apparatus operable in conjunction with a treatment apparatus configured to acquire a reference image of an anatomical region containing focal points prior to exposing the focal points to focused ultrasound (FUS) pulses and to acquire an active image for each of the focal points in the anatomical region during exposure of the focal points to the FUS pulses; and
a computing apparatus configured to interleave the reference image of the anatomical region and active images for the focal points creating a combined image of the anatomical region and the focal points, and calculate displacement measurements for the focal points exposed to the FUS pulses using the combined image.

2. A system as in claim 1, wherein the imaging apparatus comprises a Magnetic Resonance Imaging (MM) apparatus that is in communication with the computing apparatus.

3. A system as in claim 1, wherein the treatment apparatus comprises an ultrasound transducer configured to emit a pressure pattern that simultaneously focuses to two or more focal points within the anatomical region.

4. A system as in claim 1, wherein the imaging apparatus is further configured to acquire the reference image and the active images as a three-dimensional (3D) map of the anatomical region showing the focal points to be exposed to the FUS pulses.

5. A system as in claim 1, wherein the computing apparatus is further configured to monitor a temperature in the anatomical region by performing baseline subtraction between a sequence of reference images.

6. A computer implemented method for multipoint tissue displacement measurement, comprising:
generating a treatment map of an anatomical region that shows focal points within the anatomical region to be exposed to simultaneous or rapidly interleaved Focused Ultrasound (FUS) pulses;
acquiring a reference Magnetic Resonance Acoustic Radiation Force Impulse (MR-ARFI) image of the anatomical region containing the focal points using the treatment map, the reference MR-ARFI image providing a phase reference for the points to be exposed to the simultaneous or rapidly interleaved FUS pulses;
acquiring an active MR-ARFI image for each of the focal points in the anatomical region during exposure of the focal points to the simultaneous or interleaved FUS pulses using the treatment map;
interleaving the reference MR-ARFI image and active MR-ARFI images to create a combined image of the anatomical region and the focal points; and
calculating a tissue displacement measurement for the focal points exposed to the simultaneous or rapidly interleaved FUS pulses using the combined image of the anatomical region and the focal points exposed to the simultaneous or rapidly interleaved FUS pulses.

7. A method as in claim 6, wherein calculating the tissue displacement measurement further comprises using magnitude averaging for increased image Signal-to-Noise Ratio (SNR).

8. A method as in claim 6, wherein calculating the tissue displacement measurement further comprises calculating a phase for temperature and tissue displacement measurement for each focal point using a corresponding active MR-ARFI image.

9. A method as in claim 6, wherein calculating the tissue displacement measurement further comprises using displacement modeling to obtain elasticity distributions around each of the focal points.

10. A method as in claim 6, wherein calculating the tissue displacement measurement for the focal points further comprises obtaining a change in effective elasticity by combining a change in a magnitude of temperature increase and a change in displacement prior to exposing the focal points to the FUS pulses and after exposing the focal points to the FUS pulses.

11. A method as in claim 6, further comprising assessing tissue damage at the focal points according to the tissue displacement measurement as a function of FUS pulse intensity and changes in tissue displacement during exposure of tissue to the FUS pulse.

12. A method as in claim 6, further comprising determining whether a treatment endpoint has been realized based in part on the tissue displacement measurement.

13. A method as in claim 6, further comprising measuring a thermal dose delivered by the FUS pulses to tissue located at the focal points.

14. A method as in claim 6, wherein generating the map of the anatomical region further comprises:
acquiring an anatomical region MR-ARFI image of the anatomical region; and
overlaying the focal points to be exposed to FUS pulses on the anatomical region MR-ARFI image.

15. A method as in claim 6, wherein generating the map of the anatomical region further comprises generating a three-dimensional map of the anatomical region.

16. A method as in claim 6, wherein the focal points within the anatomical region mark tissue that is ablated by the FUS pulses.

17. A method as in claim 6, wherein acquiring the reference MR-ARFI image and the active MR-ARFI images further comprises using a 3D MR-ARFI spin echo technique.

18. A method as in claim 6, wherein acquiring the reference MR-ARFI image and the active MR-ARFI images further comprises using a 3D MR-ARFI gradient echo technique.

19. A method as in claim 6, wherein acquiring the active MR-ARFI image for each of the focal points in the anatomical region further comprises acquiring central slice encoding measurements for the active MR-ARFI images.

20. A method as in claim 19, further comprising acquiring outer slice encoding measurements for the reference MR-ARFI image that are interleaved with the central slice encoding measurements for the active MR-ARFI images.

21. A non-transitory machine readable storage medium having instructions embodied thereon, the instructions when executed by a processor:
generate a map of an anatomical region that shows focal points within the anatomical region to be exposed to FUS pulses;
acquire a reference MR-ARFI image of the anatomical region containing the focal points, the reference MR-ARFI image providing a phase reference for the points to be exposed to the FUS pulses;
acquire active MR-ARFI images for each of the focal points in the anatomical region during exposure of the focal points to the FUS pulses;
interleave the reference MR-ARFI image and the active MR-ARFI images to create a combined image of the anatomical region and the focal points; and
calculate displacement measurements and temperature phase measurements for the focal points exposed to the FUS pulses using the combined image of the anatomical region and the focal points exposed to the FUS pulses.
